(19) United States
(12) Patent Application Publication
(10) Pub. No.: US 2012/0000989 A1
(43) Pub. Date: Jan. 5, 2012

(54) CARTRIDGE FOR SCENT DIFFUSER, SCENT DIFFUSER AND CORRESPONDING SYSTEM

(75) Inventor: Philippe Bordier, Preaux Du Perche (FR)

(73) Assignee: BORDIER INGENIERIE, Preaux du Perche (FR)

(21) Appl. No.: 13/175,254

(22) Filed: Jul. 1, 2011

Related U.S. Application Data
(60) Provisional application No. 61/361,196, filed on Jul. 2, 2010.

(51) Int. Cl. A61L 9/04

(52) U.S. Cl. 239/34

(57) ABSTRACT

The invention concerns a cartridge to diffuse scents, comprising a hollow tubular body adapted to allow the internal circulation of air. In order to optimize the diffusion of the scent, the body comprises
At least one primary internal part in contact with the flow of air, and impregnated with an odorizing substance in order that the flow of air absorbs the perfume, and
At least one secondary internal part adjacent to the first part which allows the free circulation of the flow of air.

The invention also concerns a scent diffuser comprising a cartridge associated to a blower as well as a system comprising several diffusers and a controller.

### 1. SCOPE OF THE INVENTION

This invention concerns the diffusion of scents. More precisely, the invention concerns the controlled diffusion of scents from a scent diffuser cartridge, as well as the scent diffuser and the system comprising at least one scent diffuser.

### 2. TECHNOLOGICAL BACKGROUND

According to the state of the art, the U.S. Pat. No. 6,481,639, entitled “Volatile substance diffuser without persistence” describes scent diffusers each comprising a cartridge filled with granules of a plastic material which comprise a volatile substance such as a scent. A fan allows air to be circulated through the cartridge, the circulating air absorbing the scent and diffusing it.

This method presents the problem of insufficient diffusion of scents, for example in multimedia applications, and in particular the screening of a film in a cinema, television, games consoles, digital books with diffusion synchronized with multimedia content (for example audio and/or fixed or animated images and/or text).

### 3. SUMMARY OF THE INVENTION

The purpose of the invention is to overcome the shortcomings of previous designs.

More specifically, the objective of the invention is to optimize the diffusion of scents in synchronization with multimedia applications.

To this effect, the invention proposes a cartridge to diffuse scents. In order to optimize a diffusion of scents, the cartridge comprises a hollow tubular body, the body being adapted to allow the circulation of a flow of air within the body between an inlet and an outlet, the body comprising:
At least one primary internal part in contact with the flow of air, and impregnated with an odorizing substance in order that the flow of air absorbs the scent, and
At least one secondary internal part adjacent to the first part which allows the free circulation of the flow of air.

Furthermore, the flow of air circulates easily and absorbs the scent efficiently on contact with the primary part.

According to one preferable embodiment, the primary internal parties located at the periphery of the hollow body. The effectiveness is therefore increased, the flow of air having the tendency to concentrate around the periphery of the internal part.

According to the different embodiments, the primary part comprises a surface impregnated with the odorizing substance, and is either smooth or comprising ridges impregnated with the odorizing substance.

In another embodiment of the invention, the primary part comprises at least one cage containing elements impregnated with the odorizing substance.

According to one specific characteristic of this embodiment, at least on internal section of the hollow body will comprise a section associated to the primary part and a section associated to the secondary part, the section associated to the primary part being:
Greater than or equal to 60% of the internal section of the hollow body and
Less than or equal to 80% of the internal section of the hollow body;
   The section associated to the secondary part being:
Greater than or equal to 20% of the internal section of the hollow body and
Less than or equal to 40% of the internal section of the hollow body;

According to one specific characteristic, the cage comprises an aperture which allows it to be refilled with elements impregnated with the odorizing substance.

According to one specific characteristic, the cartridge comprises at least one gripping unit.

According to another specific, the cartridge comprises at least one valve for closure of the inlet and outlet.

According to one preferable characteristic, the length of the cartridge is greater than or equal to half of the greatest length of an internal section of the hollow body.

The invention also concerns a scent diffuser, comprising:
A cartridge as described previously, and
An air blower, adapted to pass a flow of air to an inlet in the cartridge.

According to one specific characteristic, the cartridge is removable, the scent diffuser consisting of elements which hold the cartridge in the diffuser.

According to one preferable characteristic, the blower is adapted to transfer the flow of air at a speed of between 0.5 m/s and 1.5 m/s.

According to one specific characteristic, the blower comprises a fan and the secondary part of the cartridge having an internal section of which the greatest length is between 0.8 and 1.2 times the diameter of a fan motor.

It is preferable that the blower works in conjunction with the cartridge in order that the cartridge can adopt:
A standby position, in which an inlet and an outlet for the cartridge are closed, and
An operating position, in which an inlet and outlet for the cartridge are open to allow the passage of the flow of air.

According to one specific characteristic, the diffuser comprises the means to close the aforementioned cartridge when the aforementioned cartridge is in the standby position. These means for closure are not exclusive to the aforementioned cartridge.

The invention also concerns a system for the diffusion of scents comprising at least one scent diffuser such as described previously and a least one controller adapted to control the blower associated to each scent diffuser.

### 4. LIST OF FIGURES

The invention will be better understood, and other specific features and advantages will be clarified on reading the description which follows, the description making reference to drawings in appendix, which include:

FIG. 1, which illustrates a multimedia system comprising a system for diffusion of scents comprising a scent diffuser;

FIG. 2, which illustrates a set of several scent diffusers compatible with the system in Figure;

FIGS. 3 and 4 which show two embodiments of a scent diffuser compatible with the system in FIG. 1;

FIGS. 5, 7 and 8, which illustrate cartridges designed to be used in a scent diffuser compatible with the system in FIG. 1;

FIG. 6, which shows a cross-section of the extruded olfactory material which allows the manufacture of a cartridge; and

FIGS. 9, 17 and 8, which illustrate embodiments of cartridges which can be placed in two positions in a scent diffuser compatible with the system in FIG. 1.

FIGS. 9 to 11 illustrate a front view, side view and cross section of the cylindrical cartridge.

FIG. 12 illustrates the installation of the cartridge in a mobile slot in accordance with one or more embodiments of the invention.

FIG. 13 illustrates the slot in a closed position in accordance with one or more embodiments of the invention.

FIGS. 14 and 15 illustrate the slot in which the cartridge is in operating mode.

FIGS. 16 and 17 illustrate perspective views of the slot in operating mode and standby mode.

### 5. DETAILED DESCRIPTION OF THE INVENTION

The general principal of the invention rests therefore on a scent diffuser which comprises a cartridge and a blower transferring a flow of air to an inlet in the cartridge. The cartridge is hollow to allow a flow of air and comprises a primary internal part (for example the surface and/or cage) impregnated with odorizing substances. The flow of air absorbs the scent on contact with this internal part of the cartridge. A secondary internal part of the cartridge adjacent to the primary part allows the free circulation of a flow of air and allows improved diffusion. The combined effects of the free circulation of the flow of air, and its contact with the part impregnated with an odorizing substance favors effective delivery of a flow of air containing a scent to a user.

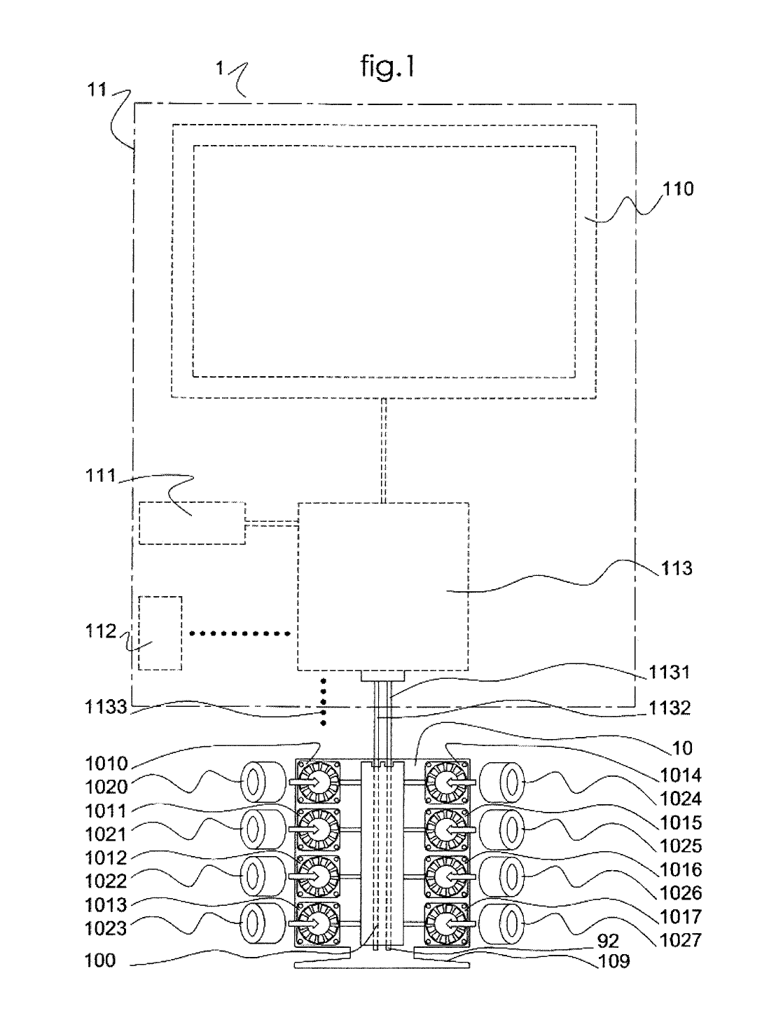

FIG. 1 illustrates a multimedia system (1) particularly well adapted to multimedia type applications, in particular video games.

System 1 comprises:
A system for diffusion of scents (10), and
An assembly of multimedia equipment (11).

The system for diffusion of scents (10) comprises:
Air blowers (1010 to 1017), associated respectively with cartridges (1020 to 1027), each adapted to a scent.
At least one controller (100) adapted to the control of the blower (1010 to 1017), and
A control unit (109)

The assembly of multimedia equipment (11) comprises a games console (113), a screen (110) connected to the games console (113), a keyboard (111) and a video games joypad (112)

The control unit (109) comprises a connector (92) (for example USB type) which allows a connection to the games console (113). This connection (for example USB type) comprises a cable (1132) to provide electrical supply to the control unit, and a cable (1131) for the transmission of commands for diffusion of scents to the controller (100). According to the embodiment, the commands are transmitted by a radio link (1133) (for example Wi-Fi or Bluetooth) to the controller (100).

The control unit (109) is for instance positioned by the screen (110) to diffuse the scents to the user of the console (100).

At a specific in a game program implemented by the console (113) (for example when the user presses one of the keys on the keyboard (111) and/or the joypad (112)), a command for the diffusion of one or several scents associated to the action is transmitted by the console (113) to the controller (100). This command is recognized by the controller (100), which activates the diffusion of the scent or scents required. Furthermore, the controller (100) activates at precise moments a diffusion of the scent of an intensity which is measured and is synchronous with the progress of the game program. The commands received by the controller (100) from the console (113) are translated in real time into signals which are sent to the air blowers (1010 to 1017). The controller (100) comprises, for example, a microcontroller or microprocessor linked to a memory comprising data which allow an identifier for the scent to be associated with an identifier for the blower (1010 to 1017). Furthermore, according to each command received from the games console requesting the diffusion of one or several scents with a corresponding intensity, the controller (100) transmits a command to the blower or blowers associated respectively to the cartridges corresponding to the scents required. To illustrate this, the cartridges (1020 and 1021) are, for example, impregnated respectively with the scents of flowers and horses. For an action associated with the smell of flowers within the game, the console (113) sends a command for the diffusion of the scent of flowers at moment t1 until moment t2, with intensity i1. Upon receiving this command containing a scent identifier and a blower identifier (in this case the blower (1010)), it tells the blower (1010) to generate a flow of air at speed s1 corresponding to intensity i1. A similar action may be repeated with another scent or combination of scents. Thus, upon receiving a command or diffusion of the smell of horses, the controller (100) tells the blower (1011) to generate a flow of air at a speed s2 corresponding to intensity i2. The controller (100) can simultaneously request several blowers if a combination of scents is required.

According to one embodiment, the commands received from the console (113) by the controller indicate an identifier to the blower (the user has, for example, placed the predetermined cartridges in a predetermined place in the control unit (109) according to instructions for the use of the game).

According to another embodiment, the cartridges (1020 to 1027) each comprise a scent identifier (for example bar-code, electronic recognition element such as an electronic component featuring an RFID identifier or label, or a mechanical selector) which can be read by the controller (100) which, according to the identifiers read, makes its memory available for another use during the diffusion of scents.

According to one embodiment, the intensity of the flow of air is constant.

According to another embodiment, the command received by the controller (100) indicates the duration of the diffusion of the scent. The controller controls the corresponding blower(s) for the required duration. According to another embodiment, the duration of the diffusion is fixed. The command for diffusion of the scent transmitted by the console 113 is then repeated according to the total duration of the diffusion of the scent required.

Furthermore, the console (113) triggers a scenario comprising a set of information representing control data. The scenario is synchronized with a video game. In this way, the console (113) can tell the controller (100) to diffuse one or several scents associated with a situation or an action in the video game.

According to an operating mode with control of the level of intensity of the scents, the control data sent by the console (113) to the controller (100) also comprises P levels of intensity, where P represents, for instance, 1, 2, 3, 4 or any value, to suit the possibilities of the air blowers (1010 to 1017) according to one predetermined linear or non-linear scale.

According to one specific operating mode, the cartridges (1020 to 1027) are specific to the game executed by the console (113).

According to one embodiment, the cartridges 1020 to 1027 are compatible with sets of scents standard to several audio/video programs or video games.

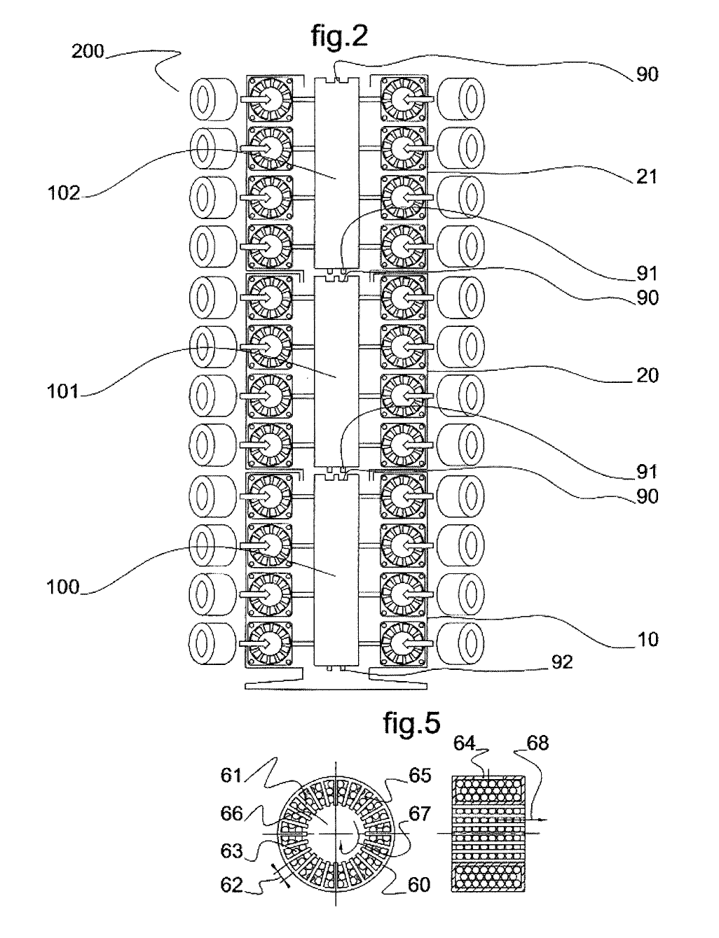

FIG. 2 illustrates a system of scent diffusers (200) which offers a large number of olfactory combinations, and comprises:
The scent diffuser control unit (10), the controller (100) being configured as the master controller
Scent control units (20 and 21) which respectively comprise controllers (101 and 102) similar to the controller (100) and configured as slave controllers

It is therefore possible to connect two, three or more diffuser control units (similar or different) in order to obtain a wider choice of scents diffused. To achieve this, each controller has a power and command input (91) and a power and command output (90) the input for one controller being connected to the output of the previous controller. By way of an example, a set of 16 connected control units each comprising eight blowers and eight cartridges makes it possible to diffuse 128 scents.

The first controller (100) or master controller manages the other controllers (101 and 102) or slave controllers. According to one particular operating mode, each controller (100 to 102) receives commands for the diffusion of a scent. It does this when an scent requested corresponds to a diffuser which it controls. It then sends the command to a controller connected to the output of its control unit. According to another operating mode, the controller (100) identifies all scent diffusers for the set of control units and controls them directly by executing the commands which it receives.

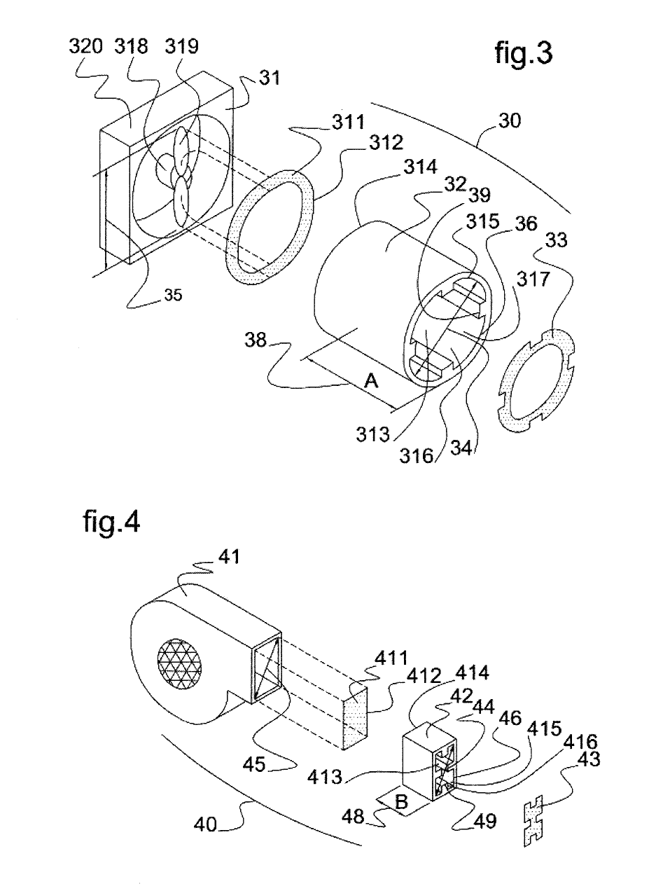

FIG. 3 presents a scent diffuser (30) comprising an axial fan type air blower (31) and an extruded cartridge (32).

The blower (31) is adapted to transfer a flow of air conducted along a section (311) to an inlet (314) in the cartridge (32). The inlet section (314) is preferably adapted to the section (311). At an outlet (315) in the cartridge (32), the flow of air exiting is conducted along a section (33).

The blower (31) is adapted to transmit a rate of flow preferably between 0.5 m/s and 1.5 m/s. Furthermore, the absorption of the scent by the flow of air is optimized when passing through the cartridge. According to other embodiments, the flow of air has a speed of less than 0.5 ms or greater than 1.5 ms.

The cartridge (32) comprises a tubular hollow section (313), adapted to allow the circulation of a flow of air to the interior of the body (313) between the inlet (31) and the outlet (315).

The hollow section (313) comprises:
A primary internal part (316) located in the periphery of the hollow section (the primary part comprises the internal face of the external envelope of the cartridge), the primary part being in contact with the flow of air and impregnated with an odorizing substance, in order that the flow of air absorbs the scent on contact with the primary part (316)
A secondary internal part (317), adjacent to the first part (316) and which allows the free circulation of the flow of air.

The odorizing substance is, for example, a synthetic or natural odorizing product (for example of animal, vegetable or mineral origin). It is preferable that the odorizing substance is compatible with the American FDA's (Food and Drug Administration) GRAS (Generally Recognized as Safe) standard.

The fan (31) comprises a motor (318), blades (319) and a housing (320). It is preferable that the secondary part (317) has an internal section which is of a greater length. Thus the power of the fan is enough to generate the flow of air required to absorb the scent: If it were shorter, the absorption of the scent would be insufficient, and if it were longer, there would be a loss of volume of the flow of air. According to one less preferable embodiment, the greatest possible length A does not fall within the range of 0.8 to 1.2 times the diameter of the motor (318).

As the blades (319) operate in a circle of diameter (35), the greatest possible length has the advantage of being at least equal to half the diameter (35). Thus, the length of the cartridge is optimized according to dimensions of the fan (31). According to one embodiment, the greatest length was less than half the diameter (35). The size of the fan and the size of the cartridge are adapted to provide the flow of scentized air required. For illustration purposes, for a flow of air which allows diffusion to a user located at a distance of 1 (one) meter, the diameter (35) of the fan, and the diameter of the cartridge (32) must be, for example, equal to 40 mm.

Preferably, the greatest internal diameter (34) of the cartridge (32) is largely equal to the diameter (35) (i.e. between 0.9 and 1.1 times the diameter (35)).

Preferably, the perimeter of the internal surface (36) of the cartridge (32) must be at least equal to the external perimeter (312) of the materialization (311) of the section for the flow of air.

The primary internal part (316) features asperities (39), for example in the form of grooves (preferably parallel to the flow of air) or protrusions. The asperities are impregnated with the odorizing substance. The addition of asperities allows the surface for the exchange of scents between the flow of air and the cartridge (32) to be increased. Preferably, the perimeter of the internal section of the cartridge (32) is equal to the perimeter of the external section of the materialization (312). It is also preferable that this perimeter is less than double the external perimeter of the materialization (312).

According to one embodiment, the internal part (316) is smooth and has no asperities (case of a cylindrical internal part (316)).

According to one development embodiment, the cartridge comprises a central part which extends into a central part along the axis of the cartridge. According to this embodiment, the central part is impregnated with an odorizing substance and the cartridge comprises an empty space around the periphery of this central part. The empty space is adjacent to this central part and allows the free circulation of a flow of air which absorbs the scent on contact with the central part.

FIG. 4 shows a scent diffuser (40) which comprises a centrifugal fan type air blower (41) and an extruded cartridge (42).

FIG. 4 shows a scent diffuser (40) comprising an axial fan type air blower (41) and an extruded cartridge (42).

The blower (41) is adapted to transfer a flow of air materialized along a section (411) to an inlet (414) in the cartridge (42). The inlet section (411) is largely rectangular and preferably adapted to the section (414). At an outlet (415) in the cartridge (42), the flow of air exiting is conducted along a section (43).

The other characteristics of the blower (41) (in particular, the speed of the flow of air) are similar to those of the blower.

The cartridge (42) comprises a tubular hollow section (413), adapted to allow the circulation of a flow of air to the interior of the body (413) between the inlet (413) and the outlet (414).

The hollow section (413) comprises:
A primary internal part (416) located at the periphery of the hollow section, in contact with the flow of air, and impregnated with an odorizing substance in order that the flow of air absorbs the scent on contact with the primary part.
A secondary internal part (417) adjacent to the first part (416) and which allows the free circulation of the flow of air.

Preferably, the greatest internal length (44) of the cartridge (42) is roughly equal to the greatest length (45) of the blower's outlet section (i.e. between 0.9 and 1.1 times the length (45).

Preferably, the perimeter of the internal surface (46) of the cartridge (42) must be at least equal to the external perimeter (412) of the materialization (411) of the section for the flow of air.

The primary internal part (416) features asperities (49) impregnated with the odorizing substance. Preferably, the perimeter of the internal section of the cartridge (42) is equal to the external perimeter of the materialization (412). It is also preferable that this perimeter is less than double the external perimeter of the materialization (412).

According to one embodiment, the internal part (416) is smooth and has no asperities (case of a cylindrical internal part (416)).

FIG. 5 shows the front view of a cartridge (65) with a longitudinal cross section. The cartridge (65) can replace the cartridge in the diffuser (30) in FIG. 3.

The cartridge (65) comprises a tubular hollow section. The body is adapted to allow the circulation of a flow of air (67 or 68) between the inlet and outlet.

A primary section of the body of the cartridge (65) comprises at least one cage (61) comprising a full and hermetic external face (60) and bars (67) with a maximum spacing (62). The cage (61) contains elements (63) impregnated with an odorizing substance. These elements are for example granules or any other element impregnated with an odorizing substance, such as:
A plastic material of a petrochemical origin, or a porous raw bio-material, with immersion or vaporization of odorizing substances, the plastic material being for example used in the form of odorizing granules.
A plastic material of a petrochemical origin, or a bio-material loaded with absorbent mineral or vegetable fibres, such as flax or hemp, with immersion or vaporization of odorizing substances, the plastic material being for example used in the form of odorizing granules.
A plastic material of a petrochemical or bio-material origin loaded with absorbent mineral or vegetable fibres, such as flax or hemp, with immersion or vaporization of odorizing substances, impregnated with odorizing substances in the form of granules and then transformed by injection or extrusion.
An absorbent paper used as a carrier for odorizing substances
A microencapsulation of odorizing substances in a housing

A secondary part (66) located at the centre of the body of the cartridge (65) is empty and allows the free circulation of the flow of air.

Preferably, the sections from the primary and secondary parts respectively represent approximately 70% and 30% of the interior of the hollow section. Thus, the circulation of air is facilitated, and the absorption by the air of the scents optimized. More precisely, the section associated to the aforementioned primary part (61) is:
Greater than or equal to 60% of the internal section of hollow body, and
Less than or equal to 80% of the internal section of the hollow body, and
   The section associated to the secondary part (66) being:
Greater than or equal to 20% of the internal section of the hollow body and
Less than or equal to 40% of the internal section of the hollow body;

According to other methods for manufacture, the section of the secondary part represents more than 40% of the internal section of the hollow body, to favor the diffusion of the air which absorbs the scent at the outlet of the cartridge.

In order to facilitate a standard manufacture of the cage of the cartridge (65), an aperture (64) allows the aforementioned cage to be refilled with elements impregnated with an odorizing substance. Thus, with a cage manufactured on a single production line, it is possible to manufacture cartridges (65) and this with specific odorizing elements. After refilling, the aperture is closed with a cover element (for example a stopper or a lid).

According to one embodiment, the cartridge (65) does not comprise an aperture. It can, for example be manufactured in two parts (for example, one part corresponding to an external face and one part with the bars, or even one front and one rear section), which can be assembled after insertion of odorizing elements.

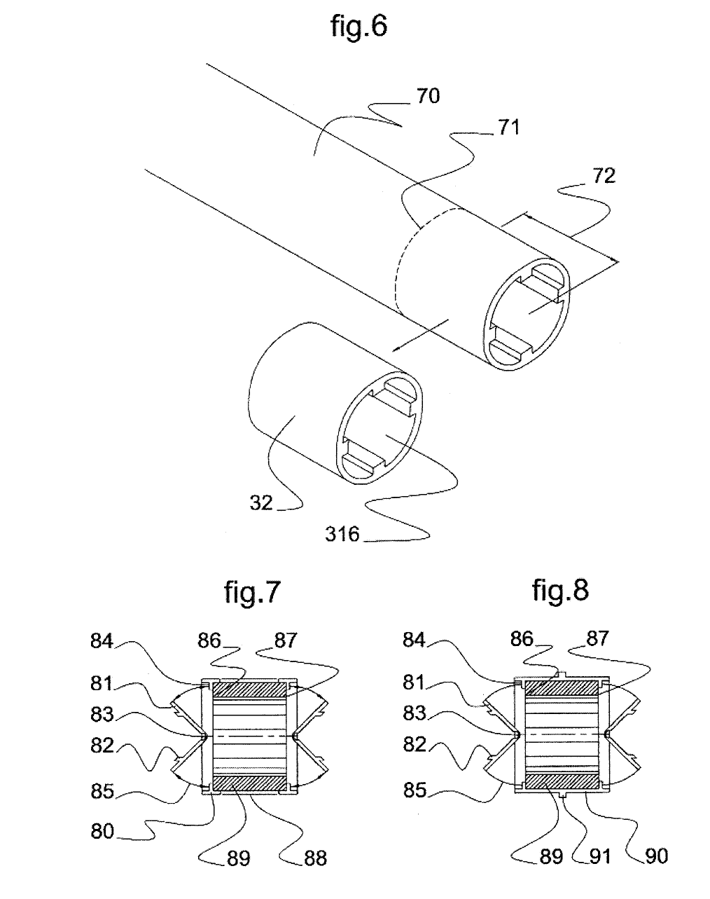

FIG. 6 illustrates a cross-section of the extruded olfactory material (70, 71, 72) which allows the manufacture of a cartridge. Specifically, FIG. 6 shows the cartridge 32 and the primary internal part (316) located in the periphery of the hollow section (the primary part comprises the internal face of the external envelope of the cartridge).

FIG. 7 illustrates a cartridge comprising a ring similar to the cartridge (32) and a mounting for the ring. The ring comprises at its extremities (86, 87) devices for sealing against light and ambient air. The articulated valves (81) comprise shapes for their gripping action (82) which pivot around an axis (83) which describe an angle (85) of up to 90°. The articulated valves (81) come into contact with the shape (84) ensuring a seal while the cartridge is not in operating mode. When it is in use, the valves (81) are held open by sprockets helping to maintain the open position. A label (88) allows the identification of a scent associated with the ring, and advises against any contact between the ring's olfactory substance (32) and the user's skin to be avoided.

According to one embodiment illustrated in FIG. 8 seals (90) replaces the label (88) and the seals (80). The seals (90) comprise blocks for guiding and holding (91) the cartridge.

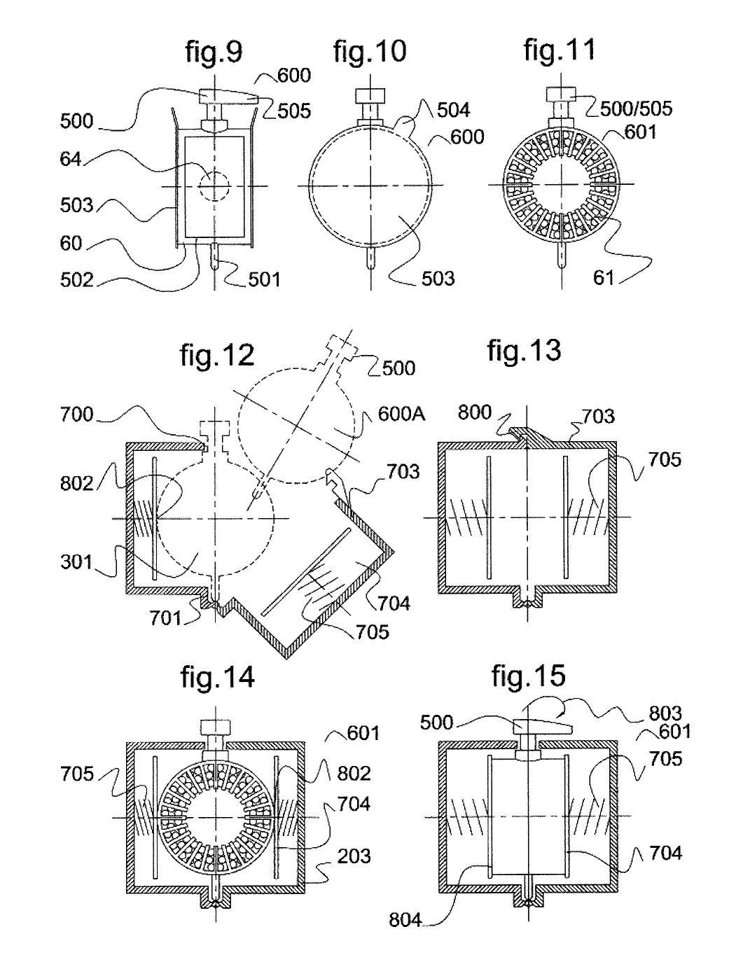

FIGS. 9 to 11 respectively, illustrate a front view, side view and cross section of the cylindrical cartridge (61). The cage (61) encloses the granules impregnated with an odorizing substance. The cartridge (600) is closed on each side by covers (503), which make it impervious to light and air prior to use. The cartridge (600) comprises a body similar to the cartridge (65) illustrated in regard to FIG. 5, a form of gripping (500) interdependent with the body of using a straight cylindrical collar and a pivot (501) on the interdependent part of the body and placed in alignment with the collar on the part opposite the collar The shape (500) comprises a protrusion (505) which allows easy manipulation. A protrusion (504) of the covers (503) provides a grip for removal of each cover.

FIG. 12 illustrates the installation of the cartridge (600) in a mobile slot (703) associated to a blower (31). The slot comprises two articulated flanges (704) which come into contact with the cartridge (600) and which are pressed against the cartridge (600) as a result of a flexible device (705) (for example in plastic or metal which acts as a spring). A hole (700) in which the collar of the part (500) locates allows the alignment of the gripping part (500) and hollow part (701) allowing the pivot (501) to be located.

FIG. 13 illustrates the slot (703) in closed position (a cartridge, not represented, can be placed inside). A quick closure (800) of the articulated slot (703) allows it to be held in the closed position.

FIGS. 14 and 15 illustrate the slot (703) in which the cartridge is respectively in operating mode (a flow of air being able to circulate in the outlet of a blower positioned opposite the cartridge) and in standby mode. The cartridge (600) pivots about a vertical axis aligned with the pivot 501. The flanges provide two balanced positions: In standby mode, the flanges hold the cartridge in a stable position while ensuring the sealing of the cartridge (600). In operating mode, the cartridge is turned through an angle of 90° about its vertical axis and the flanges hold the cartridge in another stable position: In this position, the flow of air in the outlet of a blower (31) allows the diffusion of a scent.

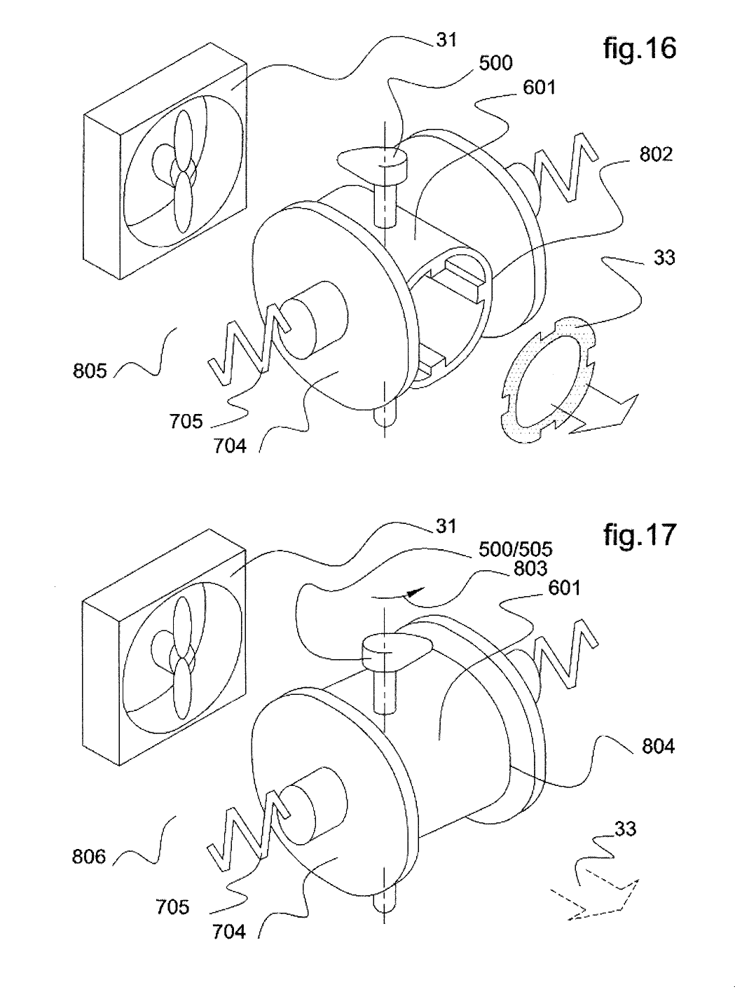

These positions are also illustrated in perspective views in FIGS. 16 and 17 for operating mode and standby mode respectively. In standby mode, an inlet and an outlet for the cartridge are closed and even if air is circulating, it will not absorb the scent. In operating mode, an inlet and outlet for the cartridge are open to allow the passage of the flow of air.

According to other embodiments, closure devices other than the flanges associated to the methods are used; in particular, the flanges can be of different forms (in particular, flat or curved) and can be rigid or flexible. Generally speaking, the closure devices are associated to a slot or devices for fixing a cartridge, the closure devices not being fixed to the cartridge.

Naturally, the invention is not limited to designs described previously.

In particular, the invention allows the diffusion of scents for diverse applications, and in particular, for cinemas, televisions, communications terminals (via internet or mobile), digital books, ambient diffusers or apparatus for medical and paramedical applications.

The skilled person can also adapt the invention to any type of mechanical structure comprising one or more scent diffusers. In particular, a system for diffusion of scents may comprise one, two, ten or more than 100 diffusers.

The invention is also compatible with various materials for the cartridges (for instance plastics, cardboard, or any other material which has a mechanical rigidity compatible with maintaining contact with a blower and a diffusion of air) or the mounting (for example metal or plastic).

The cartridges can be:
Independent, each one associated to a flow of air at the outlet of a blower, or
In a rack, with several interdependent cartridges on a mounting, each cartridge can be associated to a flow of air created by a blower (the cartridges being organized on the rack to be located opposite a flow of air when the rack is positioned for use).
   Cartridges, either in a rack or independent

According to the different embodiments of the invention, the devices for holding a cartridge in an air diffuser allow, for example, a generous adjustment (the gap and/or friction created between the cartridge and its mounting which holds the cartridge in its place) or a fast closure (the holding device comprising clips, a lock, at least one elastic device, at least one magnet and/or at least one strip of Velcro).

It should be understood that the examples and embodiments described herein are for illustrative purposes only and that various modifications or changes in light thereof can be suggested by persons skilled in the art are to be included within the spirit and purview of this application. The invention can take many other specific forms without departing from the spirit or essential attributes thereof for an indication of the scope of the invention.

What is claimed is:

1. Cartridge for scent diffuser comprising a hollow tubular body, said hollow tubular body being adapted to allow a circulation of a flow of air within said hollow tubular body between an inlet and an outlet integral to the hollow tubular body,
at least one primary internal part in contact with said flow of air, and impregnated with an odorizing substance in order that said flow of air absorbs the scent on contact with said primary internal part, and
at least one secondary internal part adjacent to said primary internal part which allows the free circulation of said flow of air.

2. Cartridge according to claim 1, wherein said primary internal part is located in a periphery of said hollow tubular body.

3. Cartridge according to claim 1, wherein said primary internal part comprises a surface impregnated with said odorizing substance.

4. Cartridge according to claim 3, wherein said surface comprises asperities impregnated with said substance.

5. Cartridge according to claim 1, wherein said primary internal part comprises at least one cage, said cage containing elements impregnated with said odorizing substance.

6. Cartridge according to claim 5, wherein at least one internal section of the hollow tubular body comprises at least one section associated with said primary internal part and a section associated with said secondary internal part, the section associated with the said primary internal part being:
greater than or equal to 60% of an internal section of the hollow tubular body, and
less than or equal to 80% of an internal section of the hollow tubular body;
the section associated to the secondary internal part being:
greater than or equal to 20% of an internal section of the hollow tubular body and
less than or equal to 40% of an internal section of the hollow tubular body;

7. Cartridge according to claim 5, wherein said at least one cage comprises an aperture allowing a filling of said at least one cage with elements impregnated with said odorizing substance.

8. Cartridge according to claim 1, wherein it comprises at least one form of gripping action.

9. Cartridge according to claim 1, wherein it comprises at least one valve for a closure of said inlet and said outlet.

10. Cartridge according to claim 1, wherein length of the cartridge is greater than or equal to half of a greatest length of an internal section of said hollow tubular body.

11. Scent diffuser, comprising:
a cartridge, and
an air blower adapted to pass a flow of air to an inlet in the said cartridge;
said cartridge comprising a hollow tubular body, said hollow tubular body being adapted to allow a circulation of the flow of air within said hollow tubular body between an inlet and an outlet integral to the hollow tubular body;
at least one primary internal part in contact with said flow of air, and impregnated with an odorizing substance in order that said flow of air absorbs the scent on contact with said primary internal part, and
at least one secondary internal part adjacent to said primary internal part which allows the free circulation of said flow of air.

12. Scent diffuser according to claim 11, wherein said cartridge is removable, said scent diffuser comprising elements to hold the cartridge in the scent diffuser.

13. Scent diffuser according to claim 11, wherein said air blower is adapted to transfer said flow of air at a speed of between 0.5 m/s and 1.5 m/s.

14. Scent diffuser according to claim 11, wherein said air blower comprises a fan and said secondary internal part of the cartridge has an internal section of which a greatest length is between 0.8 and 1.2 times a diameter of a motor for said fan.

15. Scent diffuser according to claim 11, said air blower cooperates with said cartridge to allow said cartridge to take:
a standby position, in which an inlet and an output for said cartridge are closed, and
an operating position, in which an inlet and outlet for said cartridge are open to allow a passage of the flow of air.

16. Scent diffuser according to claim 11, wherein it comprises the means to close the aforementioned cartridge when the aforementioned cartridge is in the standby position, the aforementioned device for closure not being exclusive to the aforementioned cartridge.

17. System for the diffusion of a scent comprising at least one scent diffuser, and at least one controller adapted to control the blower associated to each scent diffuser,
each scent diffuser, comprising:
a cartridge, and
an air blower adapted to pass a flow of air to an inlet in the said cartridge;
said cartridge comprising a hollow tubular body, said hollow tubular body being adapted to allow a circulation of the flow of air within said hollow tubular body between an inlet and an outlet integral to the hollow tubular body;
at least one primary internal part in contact with said flow of air, and impregnated with an odorizing substance in order that said flow of air absorbs the scent on contact with said primary internal part, and
at least one secondary internal part adjacent to said primary internal part which allows the free circulation of said flow of air.
